(19) United States
(12) Patent Application Publication
(10) Pub. No.: US 2008/0023568 A1
(43) Pub. Date: Jan. 31, 2008

(54) Discharge Device

(75) Inventors: Bjorn Weggelaar, Haarlem (NL); Casper Kleiman, Alkmaar (NL); Anton Vincent Filip Vanhoucke, Delft (NL)

Correspondence Address: YOUNG & THOMPSON, 745 SOUTH 23RD STREET, 2ND FLOOR, ARLINGTON, VA, 22202, US

(73) Assignee: SARA LEE/DE N.V., NL-3500 CA Utrecht (NL)

(21) Appl. No.: 10/590,721

(22) PCT Filed: Feb. 25, 2005
(86) PCT No.: PCT/NL05/00137

§ 371 Date: Oct. 25, 2006

(30) Foreign Application Priority Data
Feb. 27, 2004 (NL) 1025602

(51) Int. Cl. A61L 9/12; F24F 3/16

(52) U.S. Cl. 239/43; 239/60

(57) ABSTRACT

Assembly (1) of a body (5) including an agent that is in liquid or solid condition discharging an active gaseous substance, and a first holder (2) for the body, the first holder (2) having an accomodation space for accomodation therein of the body (5) and having a first wall which is part of the boundary of the accomodation space and which in a portion bounding the accomodation space is at least partially made of transparent or translucent material.

The invention relates to a discharge device for an active gaseous substance from a solid or liquid agent accommodated in a holder. Such devices are for instance used for fragrances.

Well-known is an assembly of a holder of synthetic material to be positioned upright and having a front wall and a rear wall defining an accommodation space wherein a solid body has been placed, that discharges a fragrant gas, that is able to leave the holder via apertures in the front wall of the holder. The state of the solid body in the accommodation space is hard to establish. The holder will have to be picked up and based on its weight or by shaking it an indication will have to be obtained about the size of the solid body at that moment. However, there moreover is no reference with respect to the initial state, so that no estimation can be made as to the remaining life span.

It is an object of the invention to at least improve on one of these points.

From one aspect the invention to that end provides an assembly of a body including an agent that is in liquid or solid condition discharging an active gaseous substance, and a first holder for the body, the first holder having an accommodation space for accommodation therein of the body and having a first wall which is part of the boundary of the accommodation space and which in a portion bounding the accommodation space is at least partially made of transparent or translucent material. In this way an indication regarding the (stock) state of the body accommodated in the accommodation space can be obtained in a single glance. Under circumstances the holder need not be manipulated. The stock can be timely replaced by the user.

In order to increase the perceptibility of the stock it is preferred that the portion of the first wall of the first holder bounding the accommodation space is for the larger part made of transparent or translucent material. Preferably-the portion of the first wall of the first holder bounding the accommodation space is entirely made of transparent or translucent material.

The perceptibility of the contents is optimal when the said material is transparent, preferably glass. Glass material gives the assembly a robust look.

The suitability of the assembly according to the invention in a building interior is increased when the entire first wall is made of the transparent or translucent material.

In a further development of the assembly according to the invention, the body comprises a second holder that is filled with a liquid or solid agent. The second holder can easily be separately stored, supplied and after manufacture of the first holder be placed in the accommodation space. The second holder can thus prevent pollution and thus deterioration of the transparency/translucency of the first wall of the first holder.

Preferably the first holder is suitable to be placed upright, wherein at least a lower portion of the first wall is transparent, which is particularly advantageous in case of a liquid active agent, as the last residues of the liquid will go to the lowest point and will be visible there.

In a further development the first holder has a second wall that is part of the boundary of the accommodation space. The first and the second wall can be provided with means cooperating with each other for connection to each other, which preferably are operative by means of snap action.

In order to prevent the inadvertent opening of the accommodation space—for instance when the assembly falls on the floor—and the leakage of the active agent this entails, the connection means in an embodiment constitute a connection that cannot be detached by the hands of a user.

Preferably the second wall is made of synthetic material and/or flexible material. Such a second wall, that can remain out of sight (behind the first wall) can be made cheaply, whereas the first wall can be made of a more expensive, harder material (for instance glass). The flexibility facilitates the connected to the first wall.

Preferably the second wall is provided with apertures for allowing the active substance in gaseous form through from the accommodation space. These apertures can easily be made in the synthetic material. The position of the apertures in the second wall increases the way the active agent has to cover to get to the side in front of the first wall, as a result of which said agent will have been diffused to a larger extent when it arrives there.

Diffusion of the agent can be enhanced when the second wall is concave at the side facing away from the accommodation space and at the concave side is provided with apertures for allowing the active substance in gaseous form through from the accommodation space.

In a further development the second holder has a front wall and a rear wall, which in between them define a reservoir for the active substance, wherein the front wall is at least partially, preferably at least as regards a lower portion, preferably entirely, made of transparent or translucent material, preferably transparent material. The perceptibility of the state of the stock is thus enhanced.

The rear wall can be made of a material that can be moistened by the agent in its liquid form. The agent cannot drain away through said rear wall. Yet the active substance is able to evaporate from the holder or the assembly. Particularly as a result thereof, the agent fully evaporates. The rear wall is made of a synthetic foil. The rear wall may for instance be placed in front of a second wall of the first holder which second wall is provided with apertures. In an embodiment the rear wall as well is transparent or translucent due to which the perceptibility of the stock is even further improved.

The rear wall may prior to use be sealingly closed off by a covering layer, which covering layer is provided with stripping means for the removal thereof from the rear wall and preferably is attached to a circumferential edge of the rear wall in a manner so as to be pulled loose. It is preferred here that the stripping means comprise a tab, so that removing the covering layer is easy.

Preferably the tab is connected to the covering layer at the location of an edge area thereof. The tab can thus form an integral continuation of the covering layer, so that a pulling motion can reliably be transferred to the covering layer. In an embodiment the tab can be folded against the covering layer. It is particularly preferred that in the condition in which the tab is folded against the covering layer it projects from the rear wall, so that it can be engaged in the closed condition of the accommodation space. The covering layer can then be removed in the for that matter ready-to-use condition of the assembly. Prior to use, the assembly can thus be stored in ready condition, however, without prematurely discharging active substance due to the covering layer. In an embodiment the tab is reinforced, for instance with the material of the front wall. In this way it is prevented that the tab might break.

The first holder preferably defines a passage for the tab, which passage extends from the accommodation space to the outside. In an embodiment the passage is suitable for allowing the entire covering layer including the tab through. The tab can easily be engaged and subsequently be pulled away from the first holder, thus pulling along the covering layer through the passage.

From a manufacturing point of view it is preferred that said passage is formed between the first and second wall.

Pulling and removing the tab and covering layer is enhanced when the second holder in the accommodation space defines a stop for the second holder which stop is situated near the passage.

In an embodiment the second holder is provided with a permanent lip extending through the passage in the first holder. The passage preferably is adapted such to the dimensions of the second holder that it can be moved through it. Thus the second holder can be pulled out of the first holder after use, without the first holder having to be opened.

Preferably the first holder is provided with a support surface for upright support thereof on a basis.

In an embodiment the second holder is substantially disk-shaped, preferably provided with a flange. The flange may not only serve as edge area for attachment of the covering layer, but also as placement aid for the second holder. The flange may then abut the rear side of the first wall of the first holder.

The first holder may be substantially flat.

From a further aspect the invention provides a holder for an agent, such as for instance a fragrance, that is in liquid or solid condition discharging an active gaseous substance, comprising a front wall and a rear wall, which in between them define a reservoir for the active agent, wherein the front wall is at least partially, preferably entirely, made of transparent or translucent material, preferably transparent material. The front wall preferably is made of a non-permeable material.

Preferably the rear wall is made of material that can be moistened by the agent in liquid form. In this way the active substance can be released without the agent draining away from the holder. In an embodiment the rear wall moreover is transparent or translucent. Prior to use the rear wall may be sealingly closed off by a covering layer, which covering layer is provided with stripping means for the removal thereof from the rear wall, wherein the covering layer preferably at a circumferential edge is releasably attached to the rear wall. The stripping means may comprise a tab. The tab can be connected to the covering layer at the location of an edge area thereof. The tab can foldable against the covering layer. In its condition where it is folded against the covering layer the tab may project from the rear wall. The tab may furthermore be reinforced, for instance with the material of which the front wall has been made.

The rear wall and the front wall may be attached to each other while initially leaving a fill opening free for liquid fragrance, after which the fill opening is sealed. The holder may be substantially disk-shaped, such as circular, and have a flange-shaped circumferential edge.

In an embodiment the holder has a lip by which the holder can be held. At the location of the lip the covering layer can be pre-loosened, so that the separation from the covering layer can be started there.

In a further embodiment thereof several holders are disposed in stock on a covering layer. The wanted holder can be removed from the (larger) covering layer by engaging the lip. The covering layer having several holders can be disposed on a carrier, for instance a sheet-shaped carrier.

In another embodiment several holders in stock are connected to each other by means of breakable weakening lines.

From a further aspect the invention provides an assembly of a body including an agent in liquid or solid condition discharging an active gaseous substance, and a first holder for the body, wherein the first holder has an accommodation space for accommodation therein of the body, wherein the body is formed like a second holder, having walls, which in between them define a reservoir for the active substance, preferably liquid, wherein at least one of the wells is made of a material that can be moistened by the agent in liquid form, wherein the wall prior to use is sealingly be closed off by a covering layer, which covering layer is provided with stripping means for the removal thereof from the wall, wherein the stripping means extend to beyond the first holder. Thus the covering layer can be removed when the second holder has already been accommodated in the first holder. The material that can be moistened ensures that the active substance can be released.

The stripping means may comprise a tab. The tab can be connected to the covering layer at the location of the edge area thereof. The tab can be foldable against the covering layer.

In an embodiment the said wall forms a rear wall of the second holder. The tab is then preferably attached to a circumferential edge of the rear wall so as to be pulled loose. The tab in folded condition may in that case project from the rear wall.

The first holder preferably defines a passage for the tab, which passage extends from the accommodation space to the outside. In an embodiment the passage is wide enough to also allow the entire covering layer through.

The second holder may furthermore comprise a front wall, which at least partially, preferably at least as regards a lower portion, preferably entirely, is made of transparent or translucent material, preferably transparent material. In an embodiment the rear wall as well is transparent or translucent.

The invention furthermore provides a holder suitable and intended for use as second holder in the assembly according to the invention.

From a further aspect the invention provides an assembly of a body including an agent in liquid or solid condition discharging an active gaseous substance, and a first holder for the body, wherein the first holder has an accommodation space for accommodation therein of the body, wherein the body is formed like a second holder, having walls which in between them define a reservoir for the active substance, preferably liquid, wherein at least one of the walls is made of material that can be moistened by the agent in liquid form, wherein the second holder is provided with a lip, wherein the first holder forms a passage between the accommodation space and the outer side, through which passage the lip extends, wherein the passage is dimensioned for allowing the second holder through.

From a further aspect the invention provides an assembly of a body including an agent in liquid or solid condition discharging an active gaseous substance, and a first holder for the body, wherein the first holder has an accommodation space for accommodation therein of the body, and wherein the first holder comprises two parts that can be attached to each other, which in between them define at least a part of the accommodation space, wherein the two parts are made of different material.

In an embodiment the first part is made of hard material retaining its shape and the second part is made of synthetic material.

The first part preferably is made of glass. The second part can be made of a thermoplastic synthetic material.

The second part can be provided with elastically deformable connection means for connection to the first part, such as by snapping.

The invention will be elucidated on the basis of a number of exemplary embodiments shown in the attached drawings, in which:

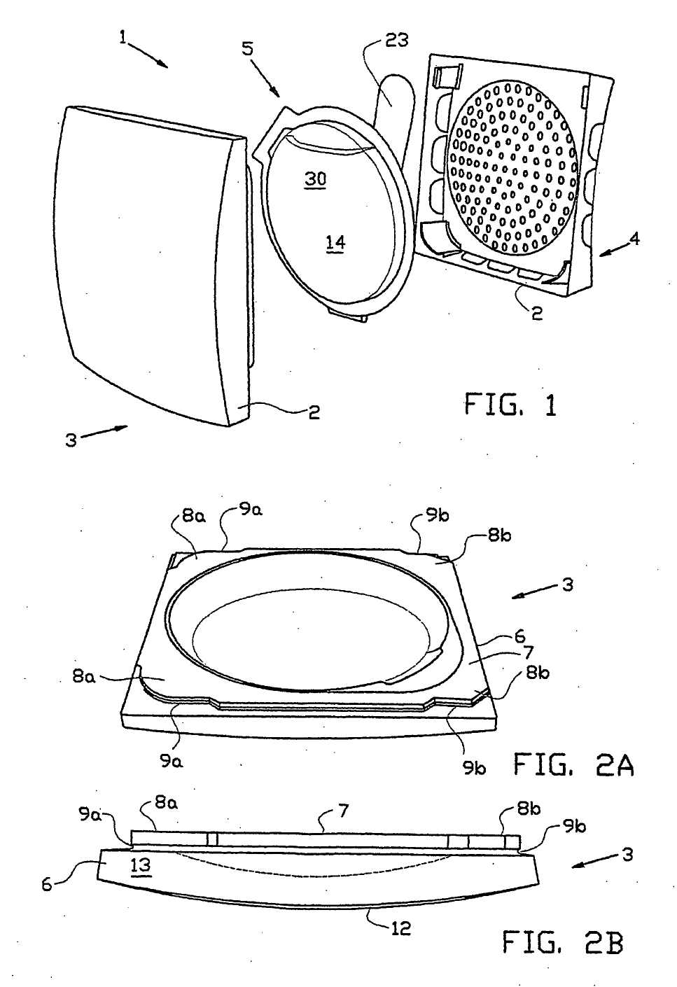

FIG. 1 shows an exploded view in perspective of an exemplary embodiment of an assembly according to the invention;

FIGS. 2A and 2B show an inclined view of the rear side and a side view of a front part, respectively, of the assembly of FIG. 1;

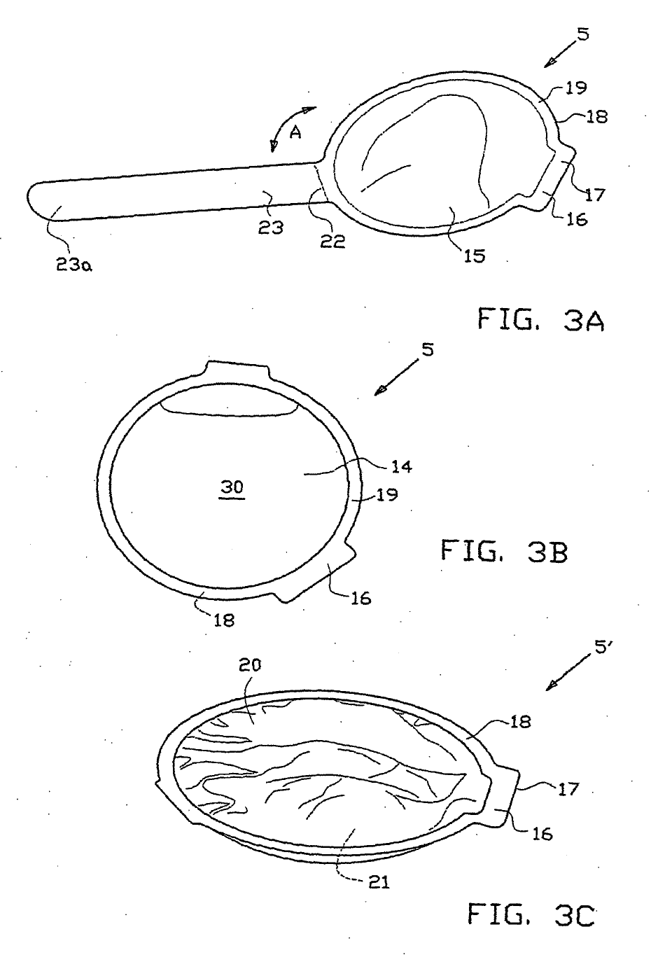

FIGS. 3A and 3B show a rear view and a front view, respectively, of a holder for a liquid fragrance-discharging agent in the assembly of FIG. 1;

FIG. 3C shows the holder of FIGS. 3A and 3B after removal of a covering layer therefrom;

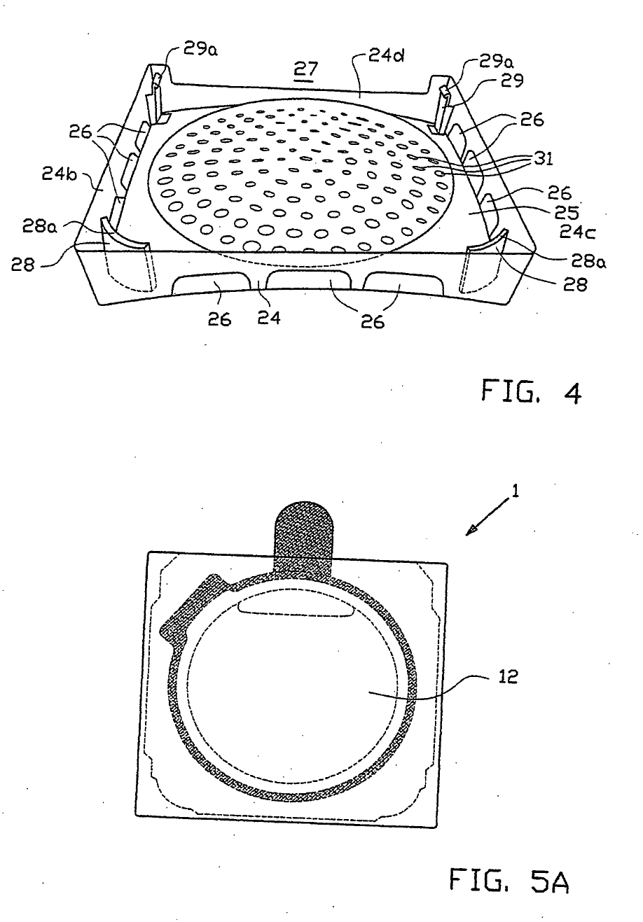

FIG. 4 shows an inclined front view of a rear part of the assembly of FIG. 1;

FIGS. 5A and 5B show a front view and a rear view, respectively, of the assembly of FIG. 1, in assembled condition;

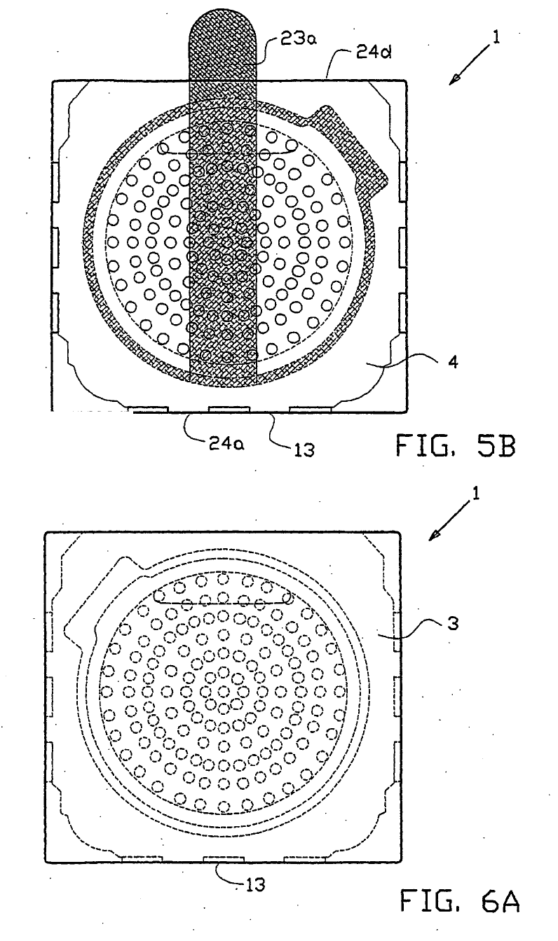

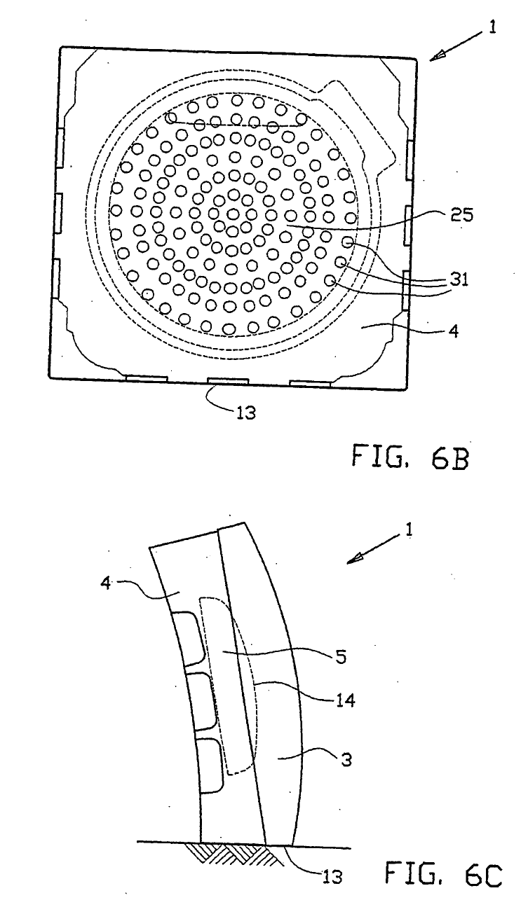

FIGS. 6A-C show a front view, rear view and side view, respectively, of the assembly of FIGS. 5A,B after removal of a covering layer therefrom;

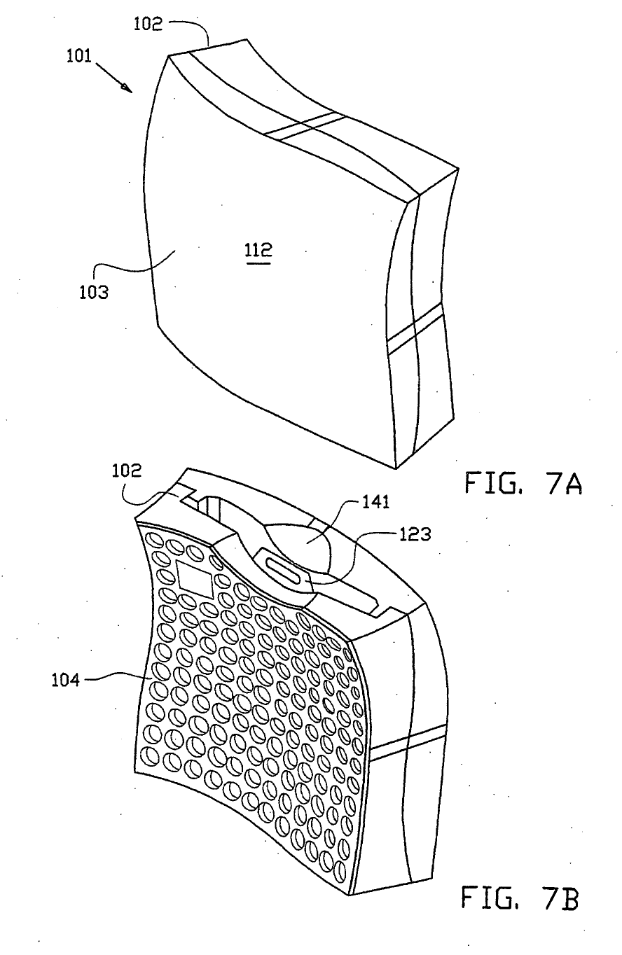

FIGS. 7A and 7B show an inclined top view and an inclined bottom view, respectively, of a second exemplary embodiment of an assembly according to the invention;

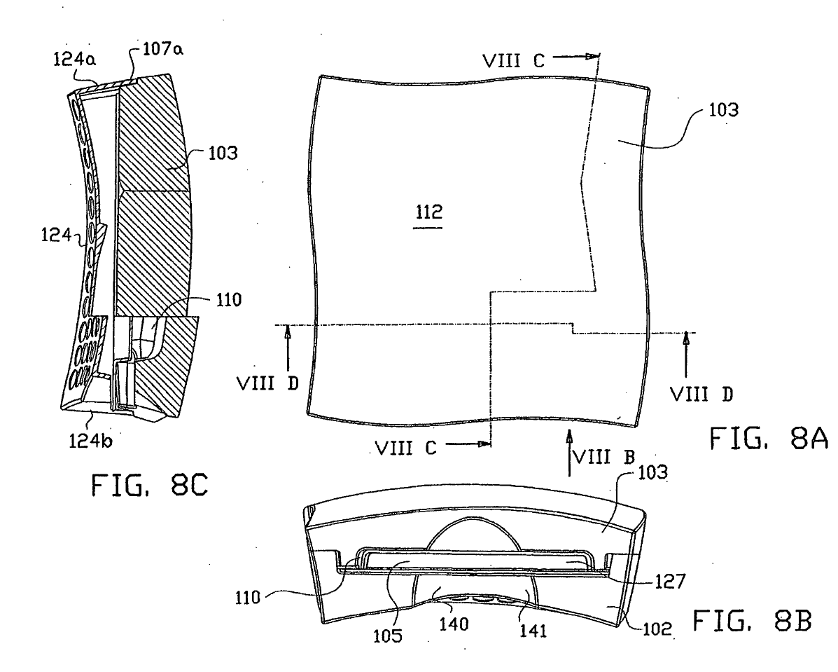

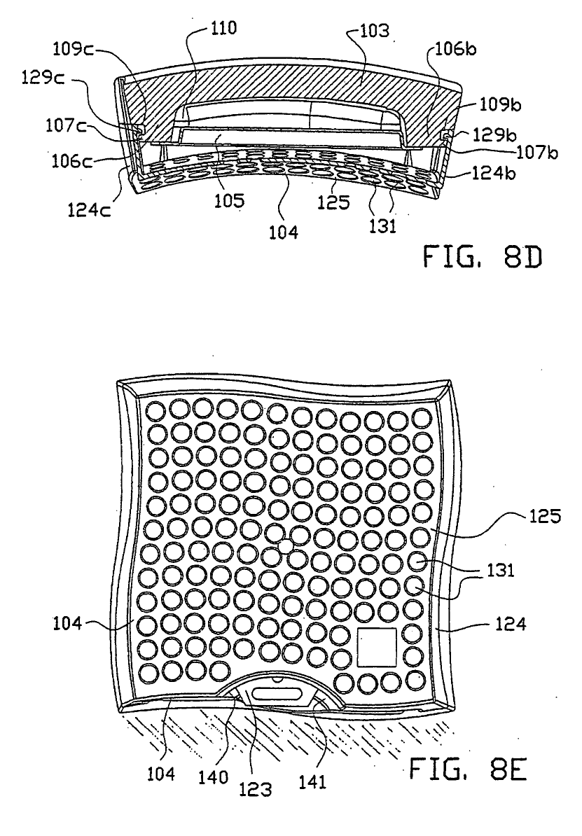

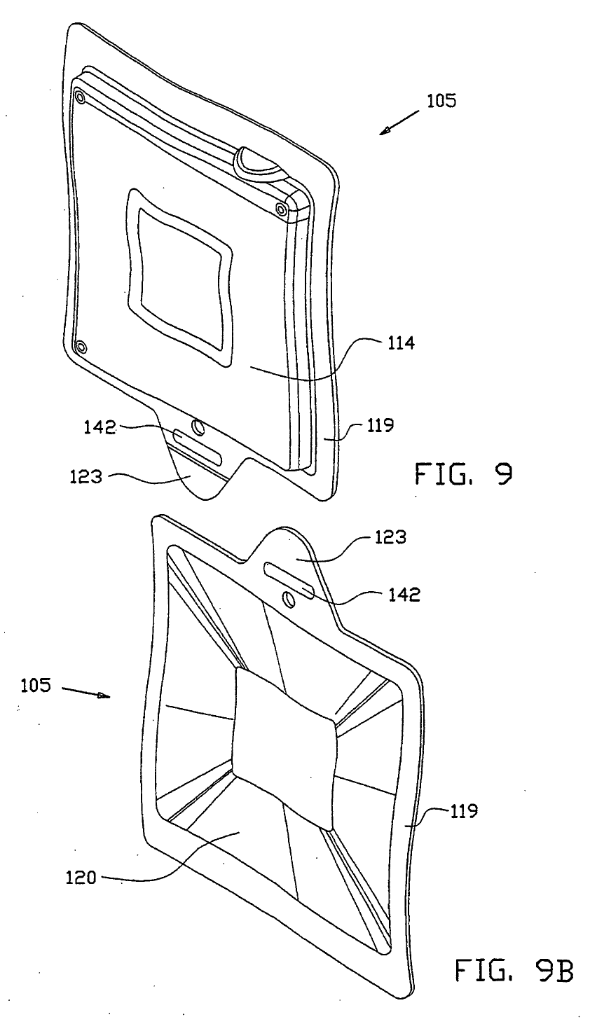

FIGS. 8A-8E show a front view, a bottom view according to VIIIB, a vertical cross-section according to VIIIC, a horizontal cross-section according to arrow VIIID and a rear view, respectively;

FIGS. 9A and 9B show a front view and a rear view, respectively, of a holder for a liquid fragrance-discharging agent in the assembly of FIGS. 7A, B;

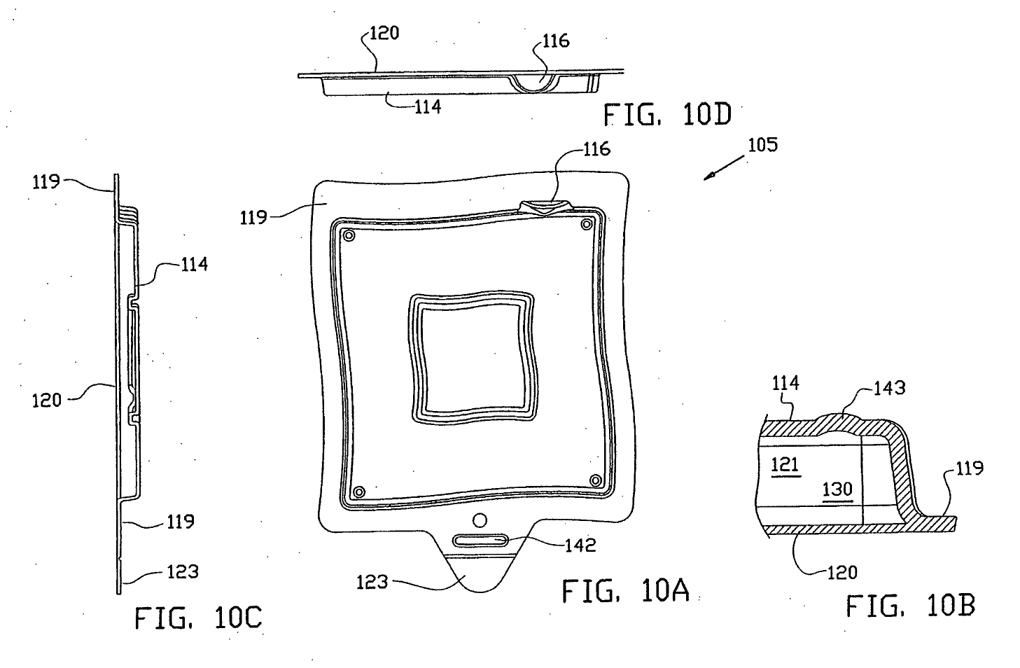

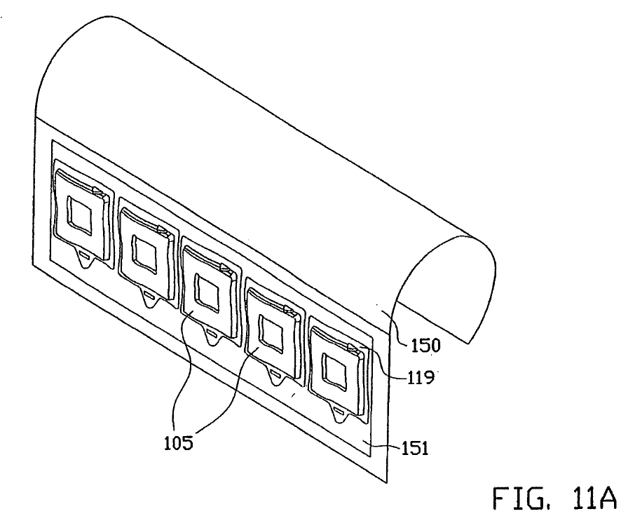

FIGS. 10A-D show a front view, a detail according to arrow XB, a cross-section according to arrow XC and a top view, respectively, of the holder of the FIGS. 9A and 9B;

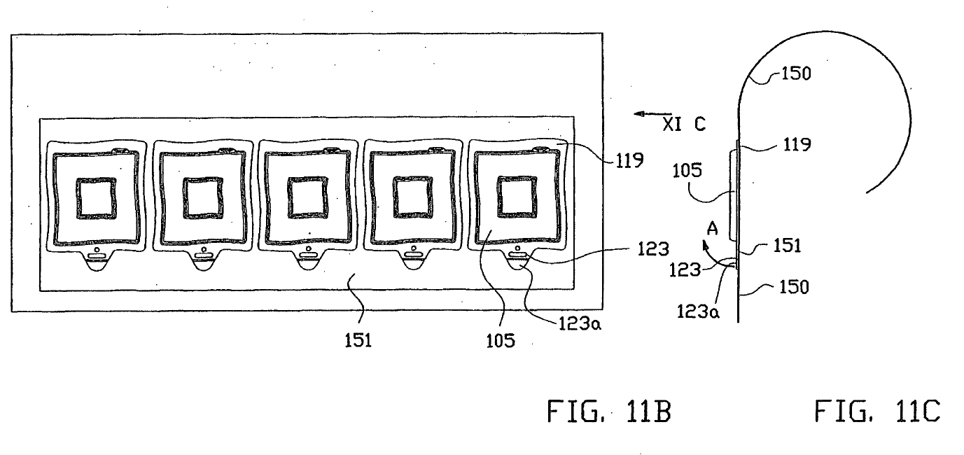

FIGS. 11A-C show a carrier having several holders according to FIGS. 9A, 9B; and

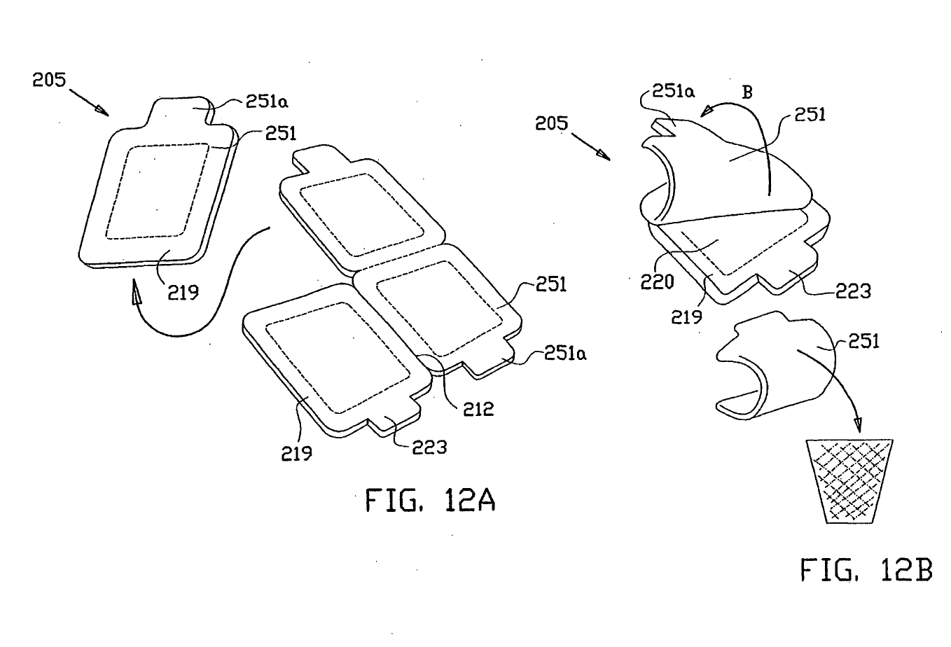

FIG. 12 shows an alternative embodiment of an assembly of several holders that are more or less similar to the holders of FIGS. 9A and 9B.

The assembly 1 shown in FIG. 1 comprises a first holder 2, in this case consisting of a front part 3 of transparent glass and a rear part 4 of translucent thermoplastic synthetic material. The front part 3 is form rigid and the rear part 4 is slightly flexible.

Between the front part 3 and the rear part 4 there is a second holder 5 for an active substance that can be discharged as a gas. In this text gas also means vaporous.

The front part 3 shown in FIGS. 2A and 2B, is substantially rectangular, in this case square, having a circumferential edge 6 situated at the rear and on which a raised circumferential border 7 has been formed, which at the location of projecting corner areas 8a,b defines a slit 9a,b with the edge 6. On the two left-hand corners, as considered in the figure, the projections 8a are arched and below it they form bent slits 9a. In the two other corners the projections 8b are straight parts forming straight slits 9b.

Within the raised circumferential border 7 there is a cavity 10, the bottom 11 of which is concave. The cavity 10 is substantially circular.

As can also be seen in FIG. 2B the edge 6 has flat sides 13 that may serve as support surface, as can also be seen in FIG. 1. It can also be seen in FIG. 2B that the front surface 12 of the front part 3 is convex.

The holder 5 shown in FIGS. 3A-C is substantially circular, adapted to the shape and dimensions of the said cavity 10. In the condition prior to use, the holder 5 is at the rear side provided with a covering layer 15 made of foil material, which along the circumferential edge 18 is sealed to a circular circumferential edge 19 of a flat bowl 14 of, in this example, impermeable transparent synthetic material. The bowl 14 defines the front wall of the holder 5.

The covering layer 15 covers off a (synthetic) membrane 20 (FIG. 3C) that can be moistened. In this example the membrane ensures that the liquid, and thus the active substance, is very gradually released and it prevents the substance from draining away. Thus an accommodation space 21 is defined between the membrane 20 and bowl 14, in which space a coloured liquid 30 is accommodated. In this example the liquid 30 is a fragrance-releasing agent.

The accommodation space 21 is filled through fill opening 16, which after filling is sealed at the location of 17. At the almost opposite side the covering layer 15 changes into a tab 23, which at the location of 22 can be folded flat against the rest of the covering layer 15 (direction A). In FIG. 3B the tab 23 is folded backwards.

The rear part 4 of the first holder 2 of the assembly of FIG. 1 is shown in FIG. 4, and comprises four sides 24a-d, wherein the sides 24a-c are provided with apertures 26 for passage of in this case a fragrance and the side 24d is provided with an opening 27 that is not bounded in forward direction.

At the location of the corners between the walls 24a,b and 24a,c raised snap lips 28 are provided, which are provided with turned end edges 28a, which are able to snap into the said slits 9a of front part 3, shown in FIG. 2A.

At the location of the corners between the walls 24b,d and 24c,d along the walls 24b,c snap lips 29 are formed, which with turned ends 29a are able to snap in the slits 9b at the other two corners of the front part 3, shown in FIG. 2A.

Between the walls 24a-d a wall 25 has been formed which is convex in forward direction and therefore is concave at the other (rear) side, and which is provided with apertures 31, like a circular surface, which as regards dimension is approximately similar to the cavity 10 in the front part 3.

In the example shown in FIG. 4, the front edges of the walls 24a-d are substantially straight, and they are situated in one plane. The edges at the other side, where the walls 24a-d change into the wall 25, are concave.

When assembling the above-mentioned parts for the assembly 1 shown in FIG. 1 into the assembly shown in FIG. 5A and further, the holder 5 is placed with the front wall 14 in the cavity 10, the covering layer 15 facing rearward. The tab 23 is then folded against the covering layer 15. With its portion 23a the tab 23 projects beyond the edge 18 and namely so far that it extends beyond the plane 13 as well. Subsequently the rear part 4 is placed by snap action of the lips 28, 29 as described above. The rear part 4 is now attached to the front part 3, while enclosing the holder 5 in the cavity 10. The covering layer 15 with tab 23 now abuts the inner surface of the wall 25. It is ensured here that the tab 23 with the end 23a extends through the opening 27.

The situation shown in FIGS. 5A and 5B is then achieved. In that condition the assembly 1 can be sold in the retail trade. Due to the transparent front part, particularly the transparent bottom 11 of the cavity 10, the transparent front wall 14 of the holder 5, the contents 30 can be properly seen. This can be helpful when making a choice when buying.

The user can easily engage the protruding part 23a of the tab 23 with two fingers and subsequently pull the tab 23, in the situation of FIG. 5A, upwards while holding the assembly 1 with the other hand. The glass front part 3 here offers a firm grip. Near the folding line 22 the covering layer 15 will be pulled loose, breaking the connection at the location of the circumferential seal 18. The foil of the covering layer 15 is pulled away while pulling onwards over 180°, and is moved upwards along the wall 25. The holder 5 itself is retained within the holder 10 due to the boundaries of said holder 10. Due to flexibility the wall 25 may be slightly resilient. When the covering layer 15 has almost been pulled halfway away, the folding line 22 arrives at the area of the opening 27. Said opening 27 is long enough (considered in circumferential direction), to allow the largest width of the covering layer 15 through. The length L of the opening 27 therefore approximately has the same size as the diameter of the holder 5/covering layer 15.

When pulling further the rest of the covering layer can easily follow, until the entire covering layer 15 has been removed from the assembly 1. What remains is the assembly shown in FIGS. 6A-C, in this example when viewed at the front (FIG. 6A), having a transparent bottom 11, a transparent front wall 14 and a rear side showing through the membrane 20, enhanced by the translucent rear wall 4. The liquid fragrance may be released as a gas or evaporate with the liquid that moistens the wall 20 that can be moistened and further evaporate through the apertures 31 in the wall 25. The assembly 1 stands firmly on a basis because of the glass front part 3.

When the liquid 30 has almost been used up this can easily be seen at the front side (FIG. 6A) and a decision can be taken whether a next assembly 1 should be bought in a particular embodiment the entire first holder is colourless transparent, and even the second holder, and the liquid is coloured. First of all, when each type of liquid has its own colour, it can immediately be seen what liquid or active substance, or what fragrance is present. Moreover the stock can be properly read.

Due to its shape the assembly can be active when upright, lying down or when suspended.

It is noted that instead of a body consisting of the second holder 5 filled with liquid, a body of a solid substance can be used. This solid body can, when the first holder 2 is closed, be surrounded by an enveloping that can be removed, for instance also by means of a tab.

The second embodiment of the assembly 101 in the FIGS. 7A, 7B and further comprises a first holder 102, in this case again consisting of a front part 103 made of transparent glass and a rear part 104 made of translucent thermoplastic synthetic material. The front part 103 is form rigid and the rear part 104 is slightly flexible. Between the front part 103 and the rear part 104 there is a second holder 105, further shown in the FIGS. 9A, 9B and further. The second holder 105 contains an active substance 30 that can be discharged as a gas.

The front part 103 is substantially square having a convex front surface 112 and at the rear side (see FIG. 8D) at the raised sides a number of side edge portions 106b,c in which ribs 107b,c and slits 109 have been formed. The edge portions 106b,c are at least situated in the lowermost portions of the vertical sides (and optionally the bottom side) of the front part 103. At the upper side of the front part 103 a notch 107a has been formed.

At the bottom side of the front part 103 a recessed wall portion 140 is present in the centre, forming a recess 141. Furthermore a flat elongates passage 127 (see FIG. 8B) is present in the same side, which passage extends from the outside to the inside, towards a cavity 110 formed in the front part 103.

In the main wall 125 the rear part 104 is provided with apertures 131. The wall 125 is formed concave.

The rear part 104 has a circumferential wall 124, wherein the vertical walls 124b, 124c and the lowermost wall 124d are provided with turned edges 129b,c,d, with which, as shown in FIG. 8D they can snap into the slits 109b,c,d formed at the location of the edge portions 106. At the upper side the wall 124a supports in the recess 107a. The rear part 104 can thus be snapped fixedly to the front part 103, optionally permanently.

The second holder 105 shown in FIGS. 9A, 9B and 10A-D is substantially square and has a front wall 114 and a rear wall 120, which are attached to each other along a circumferential flange 119. Prior to use the rear wall 120, which forms a membrane, in which for instance the liquid, and thus the active substance, is gradually released, will be covered by a covering layer of for instance foil material, that is sealed along the circumferential flange 119.

At the bottom side the flange 119 is widened with lip 123, in which a slit 142 has been arranged. Between the front wall 114 and the rear wall 120 a cavity 30 is defined, for in this example a liquid 121.

The shape and dimensions of the second holder 105 are adapted to the shape and dimensions of the cavity 110 in the first holder 102. As can be seen in the FIGS. 7A, 7B and 8A-E, the lip 123, in the condition shown wherein the second holder 105 is accommodated in the first holder 102, projects in the recess 141, and namely such that the lip 123 does not extend below the bottom side or the (stable) support surface of the first holder 102, but (after picking up the first holder 102) can indeed be engaged by the fingers of a user.

The shape and dimensions of the cross-section of the second holder 105 are adapted such to those of the passage 127, that the second holder 105 can be moved through it. In the condition of use, shown in FIG. 8E the lip 123 supporting on the basis prevents that the second holder 105 moves downward through the passage 127, so that it is indeed kept in its place in the cavity 110.

When the liquid 121 and thus the active agent in the second holder 105 has been used up, the user can pick up the assembly 101 with one hand and with the fingers of the other hand engage the lip 123 and pull the second holder 105 to the outside through the passage 127. A new second holder 105 can then be placed in reversed order.

In FIGS. 11A-C a possible arrangement of second holders 105 is shown. In this example five second holders 105 are attached to a carrier sheet 150 by means of sealing, which at one side is provided with a foil sheet 151. Said foil sheet 151 is sealed at at least the rear side of the flanges 119 of the second holders 105, so that it is ensured that the content 121, particularly the active substance of the second holders 105 is not released. A portion 123a of the lip 123 is not sealed to the foil sheet 151, and can be picked up using a nail and then a finger, in order to be bent away from the carrier sheet 150 in the direction A. By ongoing lifting of the second holder 105 the connection of the flange 119 to the foil sheet 151 is broken, and the second holder 105 is fully released in order to be inserted into the passage 127 and be slid into the first holder 102.

In FIGS. 12A and 12B an alternative way of stocking second holders 205 is shown, wherein the holders 205 are depicted slightly different from the holder 105. At the location of the edges of the flanges 219, second holders 205 are attached to each other by means of a weakening line 222, for instance perforations. Initially the flanges 219 of adjacent holders form one unity with each other. The rear wall 220 of the second holders 205 is covered with an impermeable foil 251, which is sealed circumferentially on the flanges 219.

When a new holder 205 is needed it can be separated from the series of holders by breaking the weakening lines 222. Subsequently the foil sheet portion 251a that has not been sealed to the lip 223 is taken, and it is separated from the rear wall 120 of the second holder 205 in the manner (direction B) shown in FIG. 12B. The rear wall 220 has thus become uncovered, and the second holder 205 can be slid into the first holder 102 again via the passage 127.

1-59. (canceled)

60. Assembly of a body including an agent that is in liquid or solid condition discharging an active gaseous substance, and a first holder for the body, the first holder being provided with apertures for passage of said active gaseous substance, the first holder comprising a first wall and a second wall that are provided with means cooperating with each other for connection to each other, wherein the first wall is made of hard material retaining its shape and the second wall is made of a different, synthetic and/or flexible material, said first and second walls defining an accommodation space for accommodation therein of the body, wherein said first wall in a portion bounding the accommodation space is at least partially made of transparent or translucent material.

61. Assembly according to claim 60, wherein the portion of the first wall of the first holder bounding the accommodation space is for the larger part made of transparent or translucent material.

62. Assembly according to claim 61, wherein the portion of the first wall of the first holder bounding the accommodation space is entirely made of transparent or translucent material.

63. Assembly according to claim 60, wherein the said material is transparent.

64. Assembly according to claim 60, wherein the entire first wall is made of the transparent or translucent material.

65. Assembly according to claim 60, wherein the body comprises a second holder that is filled with a liquid or solid agent.

66. Assembly according to claim 65, wherein the first holder is suitable to be placed upright, wherein at least a lower portion of the first wall is transparent.

67. Assembly according to claim 60, wherein the connection means are operative by means of snap action.

68. Assembly according to claim 60, wherein the connection means constitute a connection that cannot be detached by the hands of a user.

69. Assembly according to claim 60, wherein the second wall is provided with apertures for allowing the active substance in gaseous form through from the accommodation space.

70. Assembly according to claim 69, wherein the second wall is concave at the side facing away from the accommodation space.

71. Assembly according to claim 70, wherein the second wall at the concave side is provided with apertures for allowing the active substance in gaseous form through from the accommodation space.

72. Assembly according to claim 65, wherein the second holder has a front wall and a rear wall, which in between them define a reservoir for the active substance, wherein the front wall is at least partially made of transparent or translucent material.

73. Assembly according to claim 72, wherein the rear wall is made of a material that can be moistened by the agent in its liquid form.

74. Assembly according to claim 73, wherein the rear wall prior to use is sealingly closed off by a covering layer, which covering layer is provided with stripping means for the removal thereof from the rear wall.

75. Assembly according to claim 74, wherein the stripping means comprise a tab.

76. Assembly according to claim 75, wherein the tab is connected to the covering layer at the location of an edge area thereof.

77. Assembly according to claim 75, wherein the tab projects from the rear wall.

78. Assembly according to claim 77, wherein the first holder defines a passage for the tab, which passage extends from the accommodation space to the outside.

79. Assembly according to claim 72, wherein the second holder is provided with a permanent lip, and wherein the first holder is provided with a passage through which the lip extends.

80. Assembly according to claim 77, wherein the first holder defines a passage for the second holder, which passage extends from the accommodation space to the outside.

81. Assembly according to claim 78, wherein the passage is formed between the first and the second wall.

82. Assembly according to claim 81, wherein the passage is formed by a recess in the first wall and is further bounded by the second wall.

83. Assembly according to claim 60, wherein the first holder is provided with a support surface for upright support thereof on a basis.

84. Assembly according to claim 65, wherein the second holder is substantially disk-shaped.

85. Assembly according to claim 60, wherein the first holder is substantially flat.

86. Holder for an agent, such as for instance a fragrance, that is in liquid or solid condition discharging an active gaseous substance, comprising a front wall and a rear wall, which in between them define a reservoir for the active agent, wherein the front wall is made of transparent or translucent material, preferably transparent material.

87. Holder according to claim 86, wherein the front wall is made of a non-permeable material.

88. Holder according to claim 86, wherein the rear wall is made of material that can be moistened by the agent in liquid form.

89. Holder according to claim 88, wherein the rear wall prior to use has been sealingly closed off by a covering layer, which covering layer is provided with stripping means for the removal thereof from the rear wall, wherein the covering layer preferably at a circumferential edge is releasably attached to the rear wall.

90. Holder according to claim 89, wherein the tab is connected to the covering layer at the location of an edge area thereof.

91. older according to claim 86, wherein the holder has a lip by which the holder can be held, wherein preferably at the location of the lip the covering layer is pre-loosened.

92. Assembly of a plurality of holders according to claim 86, wherein the holders are connected to each other by means of connections that can be broken.

93. Assembly according to claim 92, wherein the rear wall is made of material that can be moistened by the agent in liquid form, preferably a synthetic membrane, wherein the rear wall preferably is transparent or translucent, wherein the rear wall prior to use has been sealingly closed off by a covering layer, which covering layer is provided with stripping means for the removal thereof from the rear wall, wherein the breakable connections are spaced apart from the tabs.

94. Assembly of a plurality of holders according to claim 86, wherein several holders are disposed in stock on a covering layer.

95. Assembly according to claim 94, wherein the covering layer with several holders is disposed on a carrier, for instance a sheet-shaped carrier.

96. Holder according to claim 86 wherein the rear wall and the front wall are attached to each other while initially leaving a fill opening free for a liquid fragrance, after which the fill opening is sealed.

97. Holder according to claim 86, wherein the holder is substantially disk-shaped, preferably rectangular.

98. Assembly of a body including an agent in liquid or solid condition discharging an active gaseous substance, and a first holder for the body, wherein the first holder has an accommodation space for accommodation therein of the body, wherein the body is formed like a second holder, having walls, which in between them define a reservoir for the active substance, preferably liquid, wherein at least one of the walls is made of a material that can be moistened by the agent in liquid form, preferably a synthetic membrane, wherein the wall prior to use is closed off by a covering layer, which covering layer is provided with stripping means for the removal thereof from the wall, wherein the stripping means extend to beyond the first holder.

99. Assembly according to claim 98, wherein the stripping means comprise a tab, wherein the tab preferably is connected to the covering layer at the location of the edge area thereof.

100. Assembly according to claim 98, wherein the tab is foldable against the covering layer.

101. Assembly according to claim 98, wherein the said wall forms a rear wall of the second holder.

102. Assembly according to claim 101, wherein the tab is attached to a circumferential edge of the rear wall so as to be pulled loose.

103. Assembly according to claim 102, wherein the tab in folded condition projects from the rear wall.

104. Assembly according to claim 98, wherein the first holder defines a passage for the tab, which passage extends from the accommodation space to the outside.

105. Assembly according to claim 98, wherein the walls of the second holder are transparent or translucent.

106. Assembly of a body including an agent in liquid or solid condition discharging an active gaseous substance, and a first holder for the body, wherein the first holder has an accommodation space for accommodation therein of the body, wherein the body is formed like a second holder, having walls which in between them define a reservoir for the active substance, preferably liquid, wherein at least one of the walls is made of material that can be moistened by the agent in liquid form, wherein the second holder is provided with a lip, wherein the first holder forms a passage between the accommodation space and the outer side, through which passage the lip extends, wherein the passage is dimensioned for allowing the second holder through.

107. Holder suitable and intended for use as second holder in the assembly according to claim 60.

108. Assembly of a body holding an agent in liquid condition or a body of an agent in solid condition discharging an active gaseous substance, and a first holder for the body, wherein the first holder has an accommodation space for accommodation therein of the body, and wherein the first holder comprises two parts that can be attached to each other, which in between them define at least a part of the accommodation space, wherein the two parts are made of different material.

109. Assembly according to claim 108, wherein the first part is made of hard material retaining its shape and the second part is made of synthetic material.

110. Assembly according to claim 109, wherein the first part is made of glass.

111. Assembly according to claim 109, wherein the second part is made of a thermoplastic synthetic material.

112. Assembly according to claim 108, wherein the second part is provided with elastically deformable connection means for connection to the first part, such as by snapping.

113. Assembly according to claim 63, wherein the said material is glass.

114. Assembly according to claim 72, wherein the front wall is at least as regards a lower portion made of transparent or translucent material.

115. Assembly according to claim 114, wherein the front wall is entirely made of transparent or translucent material.

116. Assembly according to claim 73, wherein the rear wall is made of a synthetic membrane.

117. Assembly according to claim 116, wherein the rear wall is made of a synthetic membrane and is transparent or translucent.

118. Assembly according to claim 74, wherein the covering layer is attached to a circumferential edge of the rear wall in a manner so as to be pulled loose.

119. Assembly according to claim 84, wherein the second holder is substantially disk-shaped and has a flange.

120. Holder according to claim 86, wherein the front wall is at least as regards a lower portion made of transparent or translucent material.

121. Holder according to claim 120, wherein the front wall is entirely made of transparent or translucent material.

122. Holder according to claim 88, wherein the rear wall is made of a synthetic membrane.

123. Holder according to claim 122, wherein the rear wall is made of a synthetic membrane and is transparent or translucent.

124. Holder according to claim 89, wherein the stripping means comprise a tab.

125. Holder according to claim 90, wherein the edge area forms a flange.

126. Assembly according to claim 104, wherein the first holder defines a passage for the tab, which passage extends from the accommodation space to the outside for the covering layer with tab.
